(19) United States
(12) Patent Application Publication
(10) Pub. No.: US 2005/0250091 A1
(43) Pub. Date: Nov. 10, 2005

(54) Raman molecular imaging for detection of bladder cancer

(75) Inventors: John S. Maier, Pittsburgh, PA (US); Amos Shapiro, Mevaseret-Zion (IL); Jeffrey K. Cohen, Pittsburgh, PA (US)

Correspondence Address: DUANE MORRIS, LLP;IP DEPARTMENT, 30 SOUTH 17TH STREET, PHILADELPHIA, PA, 19103-4196, US

(73) Assignee: Chemlmage Corporation, Pittsburgh, PA (US)

(21) Appl. No.: 11/000,545

(22) Filed: Nov. 30, 2004

Related U.S. Application Data
(60) Provisional application No. 60/568,357, filed on May 5, 2004.

(51) Int. Cl. C12Q 1/00; A61B 5/00

(52) U.S. Cl. 435/4; 600/315

(57) ABSTRACT

Raman molecular imaging is used to differentiate between normal tissue and benign and malignant lesions of bladder and other tissues, including epithelial tissues such as lung, prostate, kidney, breast, and colon, and non-epithelial tissues, such as bone marrow and brain. Raman scattering data relevant to the cancerous state of cells can be combined with visual image data to produce hybrid images which depict both a magnified view of the cellular structures and information relating to the cancerous state of the individual cells in the field of view.

### CROSS-REFERENCES TO RELATED APPLICATIONS

This application is entitled to priority pursuant to 35 U.S.C. §119(e) to U.S. provisional patent application 60/568,357, which was filed on 5 May 2004.

### BACKGROUND OF THE INVENTION

The invention relates generally to the field of Raman spectroscopy and mammalian cellular evaluation, including correlation of cellular physiological status and diagnosis of cancer based on such evaluation.

Cancer Diagnosis

Cancer is the second leading cause of death in the United States, with more than 1.2 million new cancers being diagnosed annually. Cancer is significant, not only in terms of mortality and morbidity, but also in terms of the cost of treating advanced cancers and the reduced productivity and quality of life achieved by advanced cancer patients. Despite the common conception of cancers as incurable diseases, many cancers can be alleviated, slowed, or even cured if timely medical intervention can be administered. A widely recognized need exists for tools and methods for early detection of cancer.

Cancers arise by a variety of mechanisms, not all of which are well understood, from evidently normal tissue. Cancers, called tumors when they arise in the form of a solid mass, characteristically exhibit decontrolled growth and/or proliferation of cells. Cancer cells often exhibit other characteristic differences relative to the cell type from which they arise, including altered expression of cell surface, secreted, nuclear, and/or cytoplasmic proteins, altered antigenicity, altered lipid envelope (i.e., cell membrane) composition, altered production of nucleic acids, altered morphology, and other differences. Typically, cancers are diagnosed either by observation of tumor formation or by observation of one or more of these characteristic differences. Because cancers arise from cells of normal tissues, cancer cells usually initially closely resemble the cells of the original normal tissue, often making detection of cancer cells difficult until the cancer has progressed to a stage at which the differences between cancer cells and the corresponding original normal cells are more pronounced. Depending on the type of cancer, the cancer can have advanced to a relatively difficult-to-treat stage before it is easily detectable.

Early definitive detection and classification of cancer is often crucial to successful treatment. Diagnosis of cancer must precede cancer treatment. Included in the diagnosis of many cancers is determination of the type and grade of the cancer and the stage of its progression. This information can inform treatment selection, allowing use of milder treatments (i.e., having fewer undesirable side effects) for relatively early-stage, non- or slowly-spreading cancers and more aggressive treatment (i.e., having more undesirable side effects and/or a lower therapeutic index) of cancers that pose a greater risk to the patient's health.

When cancer is suspected, a physician will often have the tumor or a section of tissue having one or more abnormal characteristics removed or biopsied and sent for histopathological analyses. Typically, the time taken to prepare the specimen is on the order of one day or more. Communication of results from the pathologist to the physician and to the patient can further slow the diagnosis of the cancer and the onset of any indicated treatment. Patient anxiety can soar during the period between sample collection and diagnosis.

At present there is no effective and affordable method for screening and detecting tumors in the general high-risk population. The usual test for detecting locally recurrent bladder cancer is bladder cystoscopy. Urine cytology (microscopic assessment of stained cells obtained from urine). Urine cytology is effective for detecting high-grade bladder cancers (75-90% of cases detected), but fails to detect almost all papillary urothelial neoplasms of low malignant potential. Although these low-grade cancers alone are superficial and rarely life-threatening, they can be indicative of cancer susceptibility and development. Other sensitive tests for certain low grade cancers exist (e.g., detection of markers such as BTA, Lewis X, Bard, HA-Haase, Stat, NMP 22, C-K 19, and CYFRA 21-1, and microsatellite assay), but have the disadvantages of high cost and applicability to only certain tumors. Therefore, none of these bladder cancer assays has replaced cystoscopy as a standard diagnostic procedure.

A recognized need exists to shorten the time required to analyze cells in order to determine whether or not the cells indicate the presence of cancer. Furthermore, it would be beneficial to reduce the number and/or volume of cells required for such determination, in order to minimize patient discomfort and improve patient acceptance of biopsy.

Cancer Cell Imaging Techniques

Although certain immunohistology techniques can be performed without the need for microscopic visualization of cells, almost all histopathological analysis of suspected cancer cells and tissues involves microscopic examination of the suspect cells or tissue. Optical microscopy techniques are most common, owing to their relative simplicity and the wealth of information that can be obtained by visual examination of cells and tissues.

A suspension of cells (e.g., cells in urine, blood, sputum, or a peritoneal or bronchial lavage) can be visually examined, with or without staining the suspended cells. A tissue biopsy obtained from a patient can be directly observed; stained and observed; embedded, sectioned, stained, and observed; or some combination of these.

In order to diagnose cancer, the cell or tissue preparation is analyzed by a trained pathologist who can differentiate between normal cells and malignant or benign cancer cells based on cellular morphology, tissue structure, staining characteristics, or some combination of these. Because of the tissue preparation required, this process is relatively slow. Moreover, the differentiation made by the pathologist is based on subtle morphological and other differences among normal, malignant, and benign cells, and such subtle differences can be difficult or time-consuming to detect, even for highly experienced pathologists. Such differences are even more difficult for relatively inexperienced pathologists to detect. Histopathological analysis of cell and tissue samples is highly subjective, and even experienced pathologists will sometimes differ in their assessments of the same samples. The availability of adequately trained pathologists can limit the availability or economy of screening.

Clinicians typically classify cancer lesions by assigning a grade and a stage to the lesion after superficial examination of the lesion and microscopic analysis of a biopsy taken from the lesioned tissue or organ. Grading and staging of cancers is performed by analyzing the bodily location, morphology, and extent of tissue invasion of cancer cells. The definitions of the various grades and stages of tumors vary with the type of cancer.

For solid cancers, stage describes the anatomical progression of the tumor. There are multiple systems used for staging bladder tumors. By way of example, the American Joint Committee on Cancer has set forth a bladder cancer staging system that is commonly used, known as the bladder cancer tumor-node-metastasis (TNM) staging system (“Urinary Bladder,” 1997, In: American Joint Committee on Cancer: AJCC Cancer Staging Manual., 6th ed., 2002, New York, N.Y.: Springer, 335-340). In the TNM staging system, three anatomical sites are evaluated, namely the tumor itself (T), lymph nodes (N) in the region of the tumor, and tissue sites distant from the tumor at which metastases (M) may occur. In this staging system, positive M and N scores are observed only in late stage bladder tumors, and the T score is “Ta” (non-invasive papillary carcinoma), “Tis” (carcinoma in situ), or “T1” for early stage bladder tumors. Stage scores T2, T3, and T4 in the TNM system describe successively more advanced tumors.

Grade describes the aggressiveness of the tumor cells, referring to their growth rate and likelihood of invading surrounding or distant (i.e., by metastasis) tissues. Grading is determined by microscopic analysis of tumor cells, whereby a pathologist examines how differentiated the tumor cells are from normal (non-tumorous) tissue of the same type. Tumors that resemble the corresponding normal tissue (i.e., low grade tumors) tend to grow and spread relatively slowly. In contrast, high grade tumors (i.e., those which do not resemble the corresponding normal tissue) tend to grow and spread more quickly. Patient survival is also correlated with cancer grade, higher grade corresponding to lower likelihood of survival. There are multiple systems for describing the grade of a tumors. Common systems rely on a three- or four-point grading system, the higher numbers referring to higher cancer grade. The grading system used is indicated in the grade designation, for example “I/III” referring to grade I on a three point scale and “II/IV” referring to grade II on a four-point scale.

Bladder cancer is the fifth most frequent cancer in the Western world. Early stage bladder cancer is largely asymptomatic, regardless of the aggressiveness of the cancer. Today, 85% of patients who receive an initial diagnosis of high grade bladder cancer are diagnosed with invasive bladder tumors. Such advanced stage, high grade tumors are often difficult, and sometimes impossible, to treat effectively. Accordingly, the five-year survival rate for patient diagnosed with bladder cancer is about 50%. This survival rate could be improved to 90% or better if bladder cancer could be routinely diagnosed at an early stage (e.g., at the stage of carcinoma in situ), even if only groups considered to be at high risk for developing bladder cancer (e.g., smokers over the age of 40 years and/or individuals who work in the rubber, oil, and plastic industries) were routinely screened. However, cystoscopic screening of so many individuals is impractical and would be impractically expensive.

Tools and methods for easing the cell- and tissue-based diagnoses made by pathologists would decrease the time and expense required for cancer diagnosis, grading, and staging, and would reduce the dependence of the methods on the training and expertise of the pathologist.

Raman Spectroscopy

Raman spectroscopy provides information about the vibrational state of molecules. Many molecules have atomic bonds capable of existing in a number of vibrational states. Such molecules are able to absorb incident radiation that matches a transition between two of its allowed vibrational states and to subsequently emit the radiation. Most often, absorbed radiation is re-radiated at the same wavelength, a process designated Rayleigh or elastic scattering. In some instances, the re-radiated radiation can contain slightly more or slightly less energy than the absorbed radiation (depending on the allowable vibrational states and the initial and final vibrational states of the molecule). The result of the energy difference between the incident and re-radiated radiation is manifested as a shift in the wavelength between the incident and re-radiated radiation, and the degree of difference is designated the Raman shift (RS), measured in units of wavenumber (inverse length). If the incident light is substantially monochromatic (single wavelength) as it is when using a laser source, the scattered light which differs in wavelength from the incident light can be more easily distinguished from the Rayleigh scattered light.

Because Raman spectroscopy is based on irradiation of a sample and detection of scattered radiation, it can be employed non-invasively or to analyze biological samples in situ. Thus, little or no sample preparation is required. In addition, water exhibits very little Raman scattering, and Raman spectroscopy techniques can be readily performed in aqueous environments.

Others have performed Raman spectroscopic analysis of biological tissues, including breast cancer tissue samples (e.g., Petrich, 2001, Appl. Spectrosc. Rev. 36:181; Naumann, 2001, Appl. Spectrosc. Rev. 36:239; Manoharan et al., 1998, Photochem. Photobiol. 67:15; Frank et al., 1995, Anal. Chem. 67:777; Redd et al., 1993, Appl. Spectrosc. 47:787; Haka et al., 2002, Cancer Res. 62:5375; Utzinger et al., 2001, Appl. Spectrosc. 55:955; Liu et al., 1992, Lasers Life Sci. 4:257; Frank et al., 1994, Anal. Chem. 66:319; Bakker-Schut et al., 2002, J. Raman Spectrosc. 33:580; Notingher et al., 2003, Biopolymers (Biospectroscopy 72:230-240). These investigators have used traditional Raman sampling approaches in which tissues are analyzed by collecting a Raman spectrum from a narrowly focused point in a sample.

Still other investigators (e.g., Krafft et al., 2003, Vibr. Spectrosc. 32:75-83; Kneipp et al., 2003, Vibr. Spectrosc. 32:67-74) used a Raman mapping approach wherein Raman spectra were obtained using a scanning sample holder or light source to generate a spectroscopic map of the sample. To implement this scanning strategy, there is an inherent trade off between acquisition time and the spatial resolution of the spectroscopic map. Each full spectrum takes a certain time to collect. The more spectra collected per unit area of a sample, the higher the apparent resolution of the spectroscopic map, but the longer the data acquisition takes. Performing single point measurements on a grid over a field of view will also introduce sampling errors which makes a high definition image difficult or impossible to construct. Moreover, the serial nature of the spectral sampling (i.e., the first spectrum in a map is taken at a different time than the last spectrum in a map) decreases the internal consistency of a given dataset, making the powerful tools of chemometric analysis more difficult to apply.

An apparatus for Raman Chemical Imaging (RCI) has been described by Treado in U.S. Pat. No. 6,002,476, and in co-pending U.S. Non-Provisional application Ser. No. 09/619,371, which are incorporated herein by reference. Treado disclosed that Raman molecular imaging can be used to distinguish breast cancer tissue from normal breast tissue, but did not disclose how or whether any similar method might be applicable to diagnosis, grading, or staging of bladder cancers or other cancer diagnostic methods and protocols.

The invention alleviates or overcomes the limitations of prior art tools and methods for cancer diagnosis, grading, and staging.

### BRIEF SUMMARY OF THE INVENTION

The invention relates to a method of assessing the cancerous state of a mammalian cell and the tissue from which it was derived. The method comprises irradiating the cell with substantially monochromatic light, such as laser light having a wavelength in the range from 220 to 695 nanometers. Raman scattered light emitted by the cell is assessed, for example at Raman shift (RS) values in the ranges from 280 to 1800 cm⁻¹ and from 2750 to 3200 cm⁻¹. Of course, smaller information-rich ranges, such as 500 to 1650 cm⁻¹ can be independently assessed. The intensity of the Raman scattered light emitted by the cell is compared with a reference value that corresponds to the intensity of Raman scattered light emitted by a reference cell of the same type (e.g., by a bladder cell, a kidney cell, a prostate cell, a lung cell, a colon cell, a bone marrow cell, or a brain cell). A difference between the intensity of the Raman scattered light emitted by the cell and the reference value is indicative of the cancerous state of the cell. The reference value can be a value obtained by a separate measurement performed at substantially the same time as the sample measurement, or a value stored or input into an electronic memory, for example. Preferably, the cancerous state of the reference cell is known.

It has been discovered that information indicative of cancerous state can be obtained from Raman shifted light having RS values in the ranges from 1000 to 1650 cm⁻¹ and from 2750 to 3200 cm⁻¹. Particularly informative values include RS values in the range from 1500 to 1650 cm⁻¹. The RS value of about 1584 cm⁻¹ is considered particularly informative for bladder cancer and other cancers. Other preferred RS values include RS values of about 1000, 1100, 1250, 1370, and 2900 cm⁻¹.

A variety of sources of substantially monochromatic light can be used, such as lasers (e.g., a diode pumped solid state laser). The illumination wavelength should be not greater than about 695 nanometers, and is preferably not less than about 280 nanometers. For example, a suitable laser can produce substantially monochromatic light having a wavelength of about 532 nanometers. Preferably, the bandwidth (full height at half maximum) of the substantially monochromatic light is not greater than about 0.25 nanometer.

The methods described herein can be used to assess the cancerous state of a cell either in vitro or in vivo. When in vitro analysis is performed, the cell is preferably substantially separated from red blood cells, debris, or both prior to assessing Raman scattered light emitted by the cell. By way of example, the cell can be obtained from a tissue biopsy, such as a sample of lung, bladder, prostate, kidney, colon, bone marrow, or brain tissue. The tissue can include transitional epithelium, such as that of the bladder, urethra, ureter, or kidney. The cell can also be one obtained from a body fluid such as urine, saliva, sputum, feces, blood, mucus, pus, semen, a wound exudate, or vaginal fluid. In examples described herein, analysis of bladder cells obtained from urine samples is disclosed. The cell can instead be a cell obtained from a fluid that has been contacted with a tissue of the human, such as a bladder imaging contrast fluid, a peritoneal wash fluid, a bronchial lavage fluid, a vaginal douche fluid, or a mouthwash.

When a cell is analyzed in situ in a mammal, it is necessary to use a device suitable for illuminating the cell in vivo, such as a laser illumination source that illuminates an area of a sample that includes the cell.

Instead of simply comparing a characteristic (e.g., intensity) of Raman scattered light at a single RS value, the analysis can be performed by assessing Raman scattered light emitted by the cell at two sampled RS values in the range, and comparing the ratio of intensities of the Raman scattered light emitted by the cell at the sampled RS values with a reference ratio value corresponding to the ratio of intensities of Raman scattered light emitted by a reference cell of the same type at the sampled RS values.

By making a plurality of Raman light scattering assessments of a sample (e.g., using an array of detectors in parallel), a map or image of Raman scattering information corresponding to the sample can be made. This map or image can be used by itself or combined with a visual image of the cell, optionally including its surroundings (e.g., other cells, tissues, or extracellular matrix).

The utility of the methods described herein is not limited to assessing the cancerous state of a cell. The same, or analogous, methods can be used to assess the metabolic activity, the inflammatory status, the autoimmune status, or the infected status of a mammalian cell, or the cardiac disease status of a mammalian cardiac muscle cell.

### BRIEF SUMMARY OF THE SEVERAL VIEWS OF THE DRAWINGS

FIG. 1 is a schematic diagram of an embodiment of the Raman chemical imaging system more fully described in U.S. Pat. No. 6,002,476.

FIG. 2 is a graph of Raman scattering intensity over a range of Raman shift values for bladder cells obtained from a healthy patient (thin solid and dotted lines) and for bladder cells obtained from a patient afflicted with bladder carcinoma (thick solid line). The baselines of the spectra are offset to facilitate comparison.

FIG. 3 is a graph of Raman scattering intensity over a range of Raman shift values for normal (i.e., non-cancerous) bladder tissue (dotted lines) and grade 3 transitional cell carcinoma bladder tissue (solid line).

FIG. 4 comprises FIGS. 4A, 4B, 4C, and 4D. FIG. 4A is a graph of Raman scattering intensity over a range of Raman shift values for bladder cells collected from urine of a healthy patient (thin solid line), bladder cells collected from urine of a patient afflicted with low grade (grade 1) bladder cancer (dotted line), and bladder cells collected from urine of a patient afflicted with high grade (grade 3) bladder cancer (thick solid line). The baselines of the spectra are offset to facilitate comparison. FIGS. 4B, 4C, and 4D are micrographs of a bladder cell collected from urine of a healthy patient (4B), a bladder cell collected from urine of a patient afflicted with low grade (grade 1) bladder cancer (4C), and a bladder cell collected from urine of a patient afflicted with high grade (grade 3) bladder cancer (4D).

FIG. 5 is a graph of Raman scattering intensity over a range of Raman shift values for bladder cells collected from urine of two patients afflicted with grade 2 bladder cancer (dotted lines), and bladder cells collected from urine of a patient afflicted with grade 3 bladder cancer (solid line). The baselines of the spectra are offset to facilitate comparison.

FIG. 6 is a graph of Raman scattering intensity over a range of Raman shift values for bladder cells collected from urine of three patients afflicted with grade 1 bladder cancer (five lower spectra), and bladder cells collected from urine of four patients afflicted with grade 3 bladder cancer (four upper spectra). The baselines of the spectra are offset to facilitate comparison.

FIG. 7 is a trio of averaged Raman spectra obtained from bladder cells collected from urine of normal patients (dashed line), patients afflicted with grade 1 bladder cancer (dotted line), and patients afflicted with grade 3 bladder cancer (solid line). The baselines of the spectra are offset to facilitate comparison.

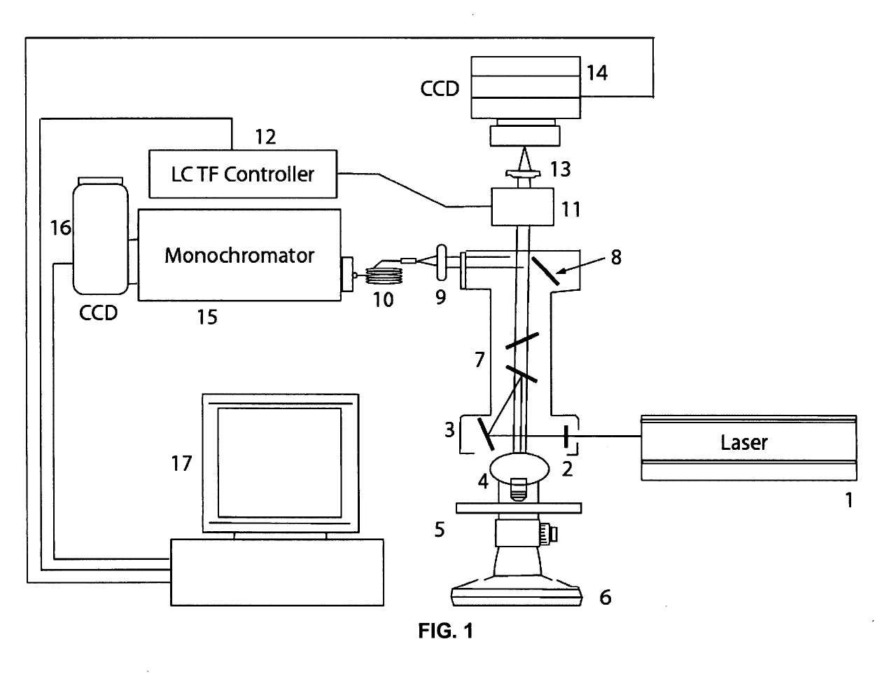

### DETAILED DESCRIPTION OF THE INVENTION

The invention relates to methods of assessing the cancerous state of a mammalian cell using a Raman spectroscopic approach. The methods are useful for assessing cells known or suspected of being cancerous, for purposes of cancer diagnosis, grading, and/or staging. The methods are useful for cancer assessment of cells of at least bladder, kidney, and breast and for detecting cells affected by infection, inflammation, autoimmune attack, and cardiac dysfunction.

The methods involve irradiating a sample including one or more mammalian cells with substantially monochromatic light and assessing Raman light scattering from the cell(s), preferably at many points on the cells in the sample or over entire areas of the sample. The intensity of Raman light scattering at one or more Raman shift values can be assessed by itself. However, a more information-rich image can be made by combining the Raman scattering data with visual microscopy data to make a hybrid image. In such an image, visual clues to the disease and/or metabolic state of the cell(s) in the sample can be derived from morphological and structural information derived from the visual microscopic image data, from the Raman scattering data, and from the superposition and/or integration of the two data sets.

The methods described herein allow quantitative evaluation of cell and tissue samples with little or no necessary sample preparation. Because the methods require relatively little cellular material, they can be performed in a non-invasive or minimally invasive manner. The methods are also suitable for in vivo or in situ use, such as with a probe inserted into a tissue or body cavity, such as the fiberscope described in U.S. Pat. No. 6,788,860.

Definitions

As used herein, each of the following terms has the meaning associated with it in this section.

“Bandwidth” means the range of wavelengths in a beam of radiation, as assessed using the full width at half maximum method.

“Bandpass” of a detector or other system means the range of wavelengths that the detector or system passes through itself, as assessed using the full width at half maximum intensity method.

The “full width at half maximum” (“FWHM”) method is a way of characterizing radiation including a range of wavelengths by identifying the range of contiguous wavelengths that over which the magnitude of a property (e.g., intensity or detection capacity) is equal to at least half the maximum magnitude of that property in the radiation at a single wavelength.

“Spectral resolution” means the ability of a radiation detection system to resolve two spectral peaks.

### DETAILED DESCRIPTION

Raman Spectroscopic Analysis for Assessment of Cancerous State

The invention is based, in part, on the discovery that certain cancer cells, when irradiated with radiation having a wavelength in the range from 220 to 695 nanometers (the wavelength preferably being greater than 280 nanometers, such as radiation having a wavelength in the range from 500 to 695 nanometers), exhibit Raman scattering of the applied radiation, and that the wavelength of the Raman scattered light emitted by those irradiated cells is shifted by amounts characteristic of the cancer cells. For example, for bladder cancer cells, characteristic RS values are in the ranges from about 1000 cm⁻¹ to 1650 cm⁻¹ and from about 2750 cm⁻¹ to 3200 cm⁻¹ (i.e., the wavelength is shifted by about 5 to 190 wavenumbers depending on illumination wavelength and the RS of interest).

In order to detect Raman scattered light and to accurately determine the Raman shift of that light, the cells should be irradiated with substantially monochromatic light, such as light having a bandwidth not greater than about 1.3 nanometers, and preferably not greater than 1.0, 0.50, or 0.25 nanometer. Suitable sources include various lasers and polychromatic light source-monochromator combinations. It is recognized that the bandwidth of the irradiating light, the resolution of the wavelength resolving element(s), and the spectral range of the detector determine how well a spectral feature can be observed, detected, or distinguished from other spectral features. The combined properties of these elements (i.e., the light source; the filter, grating, or other mechanism used to distinguish Raman scattered light by wavelength; and the detector) define the spectral resolution of the Raman signal detection system. The known relationships of these elements enable the skilled artisan to select appropriate components in readily calculable ways. Limitations in spectral resolution of the system (e.g., limitations relating to the bandwidth of irradiating light) can limit the ability to resolve, detect, or distinguish spectral features. The skilled artisan understands that and how the separation and shape of Raman scattering signals can determine the acceptable limits of spectral resolution for the system for any of the Raman spectral features described herein.

In general, the wavelength and bandwidth of light used to illuminate the sample is not critical, so long as the other optical elements of the system operate in the same spectral range as the light source. For a diffraction grating, the spectral resolution is defined as the ratio between the wavelength of interest and the separation, in the same units as the wavelength, required to distinguish a second wavelength. The apparatus described in the examples herein can distinguish a Raman shift band at 1584 cm⁻¹ from a separate peak that differs by about 12 cm⁻¹. Therefore, the Raman peak resolving power is 1584/12, or about 132, for the apparatus described in the examples. With a broader source (or a source filter enabling passage of light exhibiting an intensity profile characterized by a greater full width half maximum), greater peak separation would be required, because the Raman peaks would be more blurred on account of the greater variety of irradiating wavelengths that are shifted. Such a system would have a lower Raman peak resolving power. The lower limit in terms of preferred performance, in terms of a peak of interest at 1584 cm⁻¹ as described herein is distinguishing a peak at 1650 cm⁻¹. This represents a separation of 66 wavenumbers and indicates a preferred lower limit of Raman peak resolving power is about 1584/66=24 for these peaks. Similar calculations can be performed to determine the minimum resolving power required for distinguishing other Raman peaks described herein.

The source of substantially monochromatic light is preferably a laser source, such as a diode pumped solid state laser (e.g., a Nd:YAG or Nd:YVO₄ laser) capable of delivering monochromatic light at a wavelength of 532 nanometers. Other lasers useful for providing substantially monochromatic light having a wavelength in the range from about 280 to 695 nanometers include HeNe (630 nanometers), argon ion (532 nanometers), argon gas (360 nanometers), HeCd (442 nanometers), krypton (417 nanometers), and GaN (408 nanometers, although doped GaN lasers can provide 350 nanometers). Other lasers can be used as well, such as red diode lasers (700-785 nanometers) and eximer lasers (200-300 nanometers). Use of ultraviolet irradiation can permit use of resonance Raman techniques, which can yield more intense signals and simplified spectral peaks. However, lasers capable of ultraviolet irradiation tend to be very costly and complex to use, limiting their desirability.

Because Raman scattering peaks are independent of the wavelength of the illumination source (i.e., the RS value does not depend on the incident wavelength), the wavelength of light used to irradiate the cells is not critical. However, the illumination wavelength influences the intensity of the Raman peaks and the fluorescent background signals detected. Others have believed that irradiating cells with light having a wavelength less than those commonly used (light having a wavelength greater than about 700 nanometers is commonly used) would harm cells in the illuminated sample, owing to energy absorption by the cells.

As described herein, it has been discovered that wavelengths at least as low as about 500 nanometers (e.g., from 350 to 695 nanometers), and likely as low as 280 nanometers or even 220 nanometers, can be used without causing significant cell damage, especially if wide-field illumination techniques are employed and the intensity of the illuminating radiation is carefully controlled. Because the intensity of scattered light is known to be dependent on the fourth power of the frequency (i.e., inverse wavelength) of the irradiating light, and only proportional to the intensity of the irradiating light, lowering the wavelength of the irradiating light has the effect of increasing scattering signal output. Thus, a Raman scattering signal of equal intensity can be obtained by irradiating a sample with light having a higher wavelength and by irradiating the sample with a lower (irradiation) intensity of light having a shorter wavelength. Even under constant illumination, cells can survive irradiation with light having a wavelength as short as 500 nanometers if the intensity of the irradiating light is controlled. Irradiation using even shorter wavelengths can be performed without harming the illuminated cells if intermittent or very short duration irradiation methods are employed. Irradiating cells with sub-700 nanometer wavelength light significantly boosts the Raman scattering signal obtained from the cells, leading to greater intensity and resolution of the Raman spectra of the cells and permitting more sensitive assessment of the cancerous state of the cells than was possible using previous methods.

An appropriate irradiation wavelength can be selected based on the detection capabilities of the detector used for assessing scattered radiation. Most detectors are capable of sensing radiation only in a certain range of frequencies, and some detectors detect frequencies in certain ranges less well than they do frequencies outside those ranges. In view of the Raman shift values that can be expected from tumor tissue samples, as disclosed herein, many combinations of light sources and detectors will be appropriate for use in the systems and methods described herein. By way of example, front- and back-illuminated silicon charge coupled device (CCD) detectors are useful for detecting Raman scattered light in combination with irradiation wavelengths described herein.

A sample including one or more cells can be irradiated by the light source in a diffuse or focused way, using ordinary optics. In one embodiment, light from the source is focused on a portion of a single cell of the sample and Raman scattering from that portion is assessed. A limitation of this approach is that the power input on the illuminated area must not be so great that the cell is harmed or significantly altered, at least prior to assessment of Raman scattering. Preferably, the amount of energy transferred to the cell during illumination is not sufficient to alter the morphology, Raman spectral characteristics, or other characteristics of the cell relevant for assessment of its state.

In another embodiment, the light used to irradiate the cells is focused on a larger (i.e., whole cell or multi-cell) portion of the sample or the entire sample. Use of such wide-field illumination can diffuse the irradiation power density across the sample, reducing the rate of energy transfer to the cells therein and protecting their function and viability. Wide-field illumination allows the acquisition of data and assessment of Raman scattering across the illuminated field or, if coupled with wide-field, parallel detectors, can permit rapid Raman scattering analysis across all or part of the illuminated field. This facilitates presentation of Raman scattering data in the form of an image of all or part of the illuminated field, either alone or in combination with data obtained from the field using other spectroscopic methods. In contrast, scanning spot methods to detect Raman scattering require high laser power densities focused into a small region.

The maximum useful power density of irradiation depends on the need for post-Raman scattering assessment of the cells and the anticipated duration of irradiation. The duration and power density of irradiation must not combine to render the irradiated cells unsuitable for any desired post-assessment use. For example, when cells are irradiated in vivo, it is important that the irradiation not significantly impair the viability or biological function of the cells. In vivo irradiation should also not significantly alter the chemical signature, composition, or biological integrity of the irradiated cells and tissues. The skilled artisan is able to select irradiation criteria sufficient to avoid these effects. When prolonged irradiation of the sample is anticipated (e.g., an irradiation period minutes or hours, corresponding to a reasonable estimate of the duration of pathologist examination), the power density of illumination should be sufficiently low that the sample is not appreciably altered during the period of illumination.

If desired, the intensity of irradiation can be deliberately selected to harm or kill illuminated cells. It can be desirable to kill cancerous cells that are detected in vivo. By way of example, if a portion of the bladder epithelium of a patient is imaged using the methods described herein and portions of the epithelium are identified which harbor cancerous cells, those portions can be subjected to intense or prolonged irradiation in order to kill the cancerous cells. Alternatively, the Raman imaging methods described herein can be used to identify undesirable cells in vivo, and those undesirable cells can be ablated using a separate system which optionally employs the optics used for Raman imaging. Owing to the high resolution of the Raman scattering methods described herein, small tissue lesions can be precisely killed, even if those lesions are surrounded by or interspersed with regions of healthy tissue. Thus, for example, these methods can be used to direct destruction of cancerous cells in an epithelium.

Imagographic analysis of Raman scattering on a cellular scale can be performed using known microscopic imaging components. High magnification lenses are preferred, owing to their higher light collection relative to low magnification lenses. The numerical aperture of the lens determines the acceptance angle of light into the lens, so the amount of light collected by the lens varies with the square of the numerical aperture. By way of example, a 100× objective lens will typically have a numerical aperture value of about 0.9, and most 20× objective lenses will have a numerical aperture of about 0.4. Thus, the amount of light collected by the 100× lens will be about five times greater than the amount of light collected by the 20× lens. In view of the fact that Raman scattered light can have a relatively low magnitude, selection of a high magnification lens can improve low level signal detection.

Assessment of Raman scattered light can be measured using any known detector appropriate for sensing radiation of the expected wavelength (generally about 5 to 200 nanometers greater than the wavelength of the irradiating radiation). In view of the relatively low intensities of many Raman scattered light signals, a highly sensitive detector may be preferred or required, such as one or more cooled charge-coupled device (CCD) detectors. For parallel operation, CCD detectors having multiple pixels corresponding to discrete locations in the field of illumination can be used to enable simultaneous capture of spectroscopic data at all pixel locations in the CCD detector.

Raman scattered light can be assessed at individual points in a sample, or an optical image of the Raman scattered light can be generated using conventional optics. The Raman data or image can be visually displayed alone or in combination with (e.g., superimposed upon) a microscopic image of the sample. Conventional methods of highlighting selected Raman data (e.g., by color coding or modulating the intensity of Raman scattered light) can be used to differentiate Raman signals arising from various parts of the sample. By way of example, the intensity of Raman scattered light having a Raman shift of 1584 cm⁻¹ can be displayed in varying shades or intensity of green color, superimposed on a brightfield optical microscopic image of the sample. In this way, Raman scattering can be correlated with microscopic landmarks in the sample.

If the cells are irradiated using light having a wavelength in the range from about 500 to 700 nanometers, then an RS value in the range 1000 to 1650 cm⁻¹ can be assessed using a detector capable of detecting radiation having a wavelength of about 550 to 785 nanometers, and an RS value in the range 2750-3200 cm⁻¹ can be assessed using a detector capable of detecting radiation having a wavelength of about 650 to 890 nanometers.

Cells include many chemical species, and irradiation of cells can result in Raman scattering at a variety of wavelengths. In order to determine the intensity of Raman scattered light at various RS values, scattered light corresponding to other RS values must be filtered or directed away from the detector. A filter, filter combination, or filter mechanism can be interposed between the irradiated sample and the detector to accomplish this. The system (i.e., taking into account the bandwidth of the irradiating radiation and the bandpass of any filter or detector) should exhibit relatively narrow spectral resolution (preferably not greater than about 1.3 nanometers, and more preferably not greater than about 1.0, 0.5, or 0.25 nanometers) in order to allow accurate definition and calculation of RS values for closely spaced Raman peaks. If selectable or tunable filters are employed, then they preferably provide high out-of-RS band rejection, broad free spectral range, high peak transmittance, and highly reproducible filter characteristics. A tunable filter should exhibit a spectral resolving power sufficient for Raman spectrum generation (e.g., a spectral resolving power preferably not less than about 12-24 cm⁻¹). Higher and lower values can be suitable, depending on the bandwidth of irradiating radiation and the Raman shift values desired to be distinguished.

A tunable filter is useful when Raman scattering measurements are simultaneously made at multiple locations in the illuminated field and when a Raman spectrum (i.e., assessments at multiple RS values such as assessments at sequential RS values made at 20 cm⁻¹ increments) is to be obtained using the detector (e.g., for collecting 2-dimensional RS data from a sample). A variety of filter mechanisms are available that are suitable for these purposes. For example, an Evans split-element liquid crystal tunable filter (LCTF) such as that described in U.S. Pat. No. 6,002,476 is suitable. An LCTF can be electronically controlled to pass a very narrow wavelength band of light. The spectral resolving power of 8 cm⁻¹ (0.25 nanometer) is suitable to perform Raman spectroscopy, and the image fidelity is sufficient to take full advantage of the resolving power of a light microscope, yielding a spatial resolution of better than 250 nanometers. Other suitable filters include Fabry Perot angle-rotated or cavity-tuned liquid crystal (LC) dielectric filters, other LC tunable filters (LCTF) such as Lyot Filters and variants of Lyot filters including Solc filters, acousto-optic tunable filters, and polarization-independent imaging interferometers such as Michelson, Sagnac, Twynam-Green, and Mach-Zehnder interferometers. This list of suitable filters is not exhaustive.

Raman Scattering by Bladder Cancer Cells

The invention includes the discovery that bladder cancer cells exhibit significant Raman scattering at an RS value of about 1584 cm⁻¹, relative to non-cancerous bladder cells. Furthermore, the intensity of Raman scattering at this RS values increases with increasing grade of bladder cancer. Other RS values at which Raman scattering is associated with the cancerous state of bladder cells include about 1000, 1100, 1250, 1370, and 2900 cm⁻¹. This list of values is not exhaustive. Furthermore, there is a generalized increase in Raman scattering at RS values in the range from about 1000 to 1650 cm⁻¹ and in the range from about 2750 to 3200 cm⁻¹ in bladder cancer cells, relative to non-cancerous bladder cells, and this generalized increase is more pronounced in the range of RS values from about 1530 to 1650 cm⁻¹. These RS values and ranges are useful for assessing the cancerous state of bladder cancer cells.

Scattering intensity values assessed at one or more RS values can be correlated with cancerous state of the corresponding cell(s) by observing the existence (or non-existence) of increased RS intensity relative to a non-cancerous cell (or to a cell exhibiting a lower grade of cancer. This assessment can be performed using raw intensity values, by comparing intensities at different parts of a sample (e.g., portions that exhibit distinct morphological appearances), by comparing intensity at multiple RS values (e.g., comparing the values of the ratio of intensity at RS value 1584 cm⁻¹ with the intensity at an RS value that does not vary significantly with cancerous state, such as RS value 1450 cm⁻¹), by combining analysis of an RS value with light microscopy information, or by other methods apparent to one skilled in Raman spectroscopy, pathology, visible light microscopy, or some combination of these disciplines.

The ratio of Raman scattering intensities at two RS values can vary at different irradiation wavelengths, but will normally exhibit similar trends. This variation is attributable to the nature of Raman scattering. Raman scattering at a particular RS value depends on both the electronic and vibrational structure of the illuminated molecule. Ordinarily, the electronic state of the molecule does not affect Raman scattering, and electronic and vibrational structures are often considered independent of one another to simplify understanding. Sometimes, however, the energy of illuminating radiation can be used to shift the electronic state of the illuminated molecule, and the transition of the molecule between electronic states resonantly enhances vibration of the molecule. The result of these processes is a very significant (e.g., 100- to 1000-fold) increase in the intensity of the scattered radiation. This enhanced scattering intensity is commonly called resonance Raman scattering and can greatly simplify signal detection, especially in noisy backgrounds. By varying the wavelength of light used to illuminate a cell, resonance Raman effects can be avoided or taken advantage of (e.g., depending on whether the resonating molecule corresponds to an RS value that is informative regarding the cancerous state of a bladder cell or not).

The methods described herein can be used by assessing the intensity of light scattered from a portion of the sample and subtracting out the intensity of light scattered from a different, reference portion of the sample that is known or believed to correspond to normal (i.e., non-cancerous) tissue. For example, RS data from different cells or from different areas of a single tissue sample can be compared. A difference of scattered light intensity between the analyzed and reference portions of the sample indicates a difference in cancerous state.

Tissues sometimes exhibit localized fluorescence which, if not accounted for, can complicate cancer cell assessment. If such fluorescence occurs at a wavelength of interest for assessing the cancerous state of a cell in a sample, then a subtractive method can be used to correct for tissue fluorescence and prevent fluorescent emissions from obscuring relevant scattering data.

In general, fluorescent emission is spectrally much broader than Raman scatter. For instance a typical Raman band in a bladder cancer sample will have a bandwidth of about 20 cm⁻¹. In contrast, the fluorescence spectrum (which can be tens to hundreds of nanometers in breadth) of the same bladder cancer sample irradiated with the same light will have a bandwidth of thousands of wavenumbers. Because of this, strategic choices of where in Raman shift space measurements are made (i.e., choice of which RS values are used for scattered light intensity measurements) permit correction for fluorescent emissions. By way of example, two image frames can be assessed in Raman space, one at 1584 cm⁻¹ and another at 2600 cm⁻¹. The radiation detected in the frame assessed at 2600 cm⁻¹ will consist essentially of radiation fluorescently emitted from the sample, because there is essentially no Raman scatter at this RS value. The radiation detected in the frame assessed at 1584 cm⁻¹ will include both Raman scattered radiation and fluorescently emitted radiation having substantially the same intensity as radiation fluorescently emitted at 2600 cm⁻¹. Subtracting the intensity of emissions assessed at 2600 cm⁻¹ from the intensity of emissions at 1584 cm⁻¹ will yield an intensity value essentially equal to the intensity of Raman scattered light at 1584 cm⁻¹. This is one example of a way by which the intensity of Raman scattered light from a sample can be assessed even if the sample also fluorescently emits light having the same wavelength as the Raman shifted light.

Materials present in cell and tissue samples obtained from humans or other mammals can interfere with Raman scattering of the cells of interest in the samples. These materials are preferably removed prior to Raman scattering analysis of the cells. By way of example, red blood cells (RBC) exhibit strong Raman scattering at RS values including or overlapping 1581 cm⁻¹, and debris such as that which commonly occurs in bodily fluids or in excised samples can exhibit Raman scattering at a wide variety of RS values. RBC and debris can be removed from samples in relatively straightforward ways using known methods, such as gently rinsing cell samples with distilled, deionized water, normal saline, an acetic acid solution, or dilute phosphate buffer. For example, combining a urine sample with an acetic acid solution will induce rupture of RBCs, leaving other cells intact, as is known.

Raman Scattering by Cells Other than Bladder Cancer Cells

It was discovered that diseased cells other than cancerous bladder cancer cells exhibit enhanced Raman scattering at the RS values disclosed herein, relative to the corresponding non-diseased cells. Examples of diseased cells which can be differentiated from non-diseased cells of the same type using the methods described herein include cancerous kidney cells (e.g., renal tubular cells), cancerous prostate cells, cancerous colon cells, cancerous breast cells, cancerous lung cells, cancerous bone marrow cells, cancerous brain cells, cells of inflamed tissues, cells of tissues undergoing autoimmune attack, and cardiac muscle cells of diseased heart tissue (e.g., ischemic heart tissue). The methods described herein can be used to assess the diseased state of cells of at least these types by assessing Raman scattering by the cells at RS values in the range 1000 to 1650 cm⁻¹ and/or 2750-3200 cm⁻¹ or at the particular RS values indicated herein. Comparison of Raman scattering at those RS values with reference values or with non-diseased cells of the same type can indicate the diseased state of the sampled tissue.

Without being bound by any particular theory of operation, it is believed that tissues for which a diseased state can be detected using the methods described herein exhibit altered metabolic activity, relative to corresponding non-diseased cells of the same type. The altered metabolic activity is thought to be attributable to one or more disease processes occurring in the tissue. For instance, in an inflamed tissue or organ, an altered metabolic activity is required to mount the inflammatory response to the inciting event. This response drives the cells/tissues into a state of altered metabolic activity, further altering the biochemical makeup of the cell. These alterations are manifested in the Raman scattering characteristics of the cells or tissues. An implication of this theory of operation is that the methods disclosed herein should be useful for differentiating diseased and non-diseased tissue for many (and potentially all) diseases that are characterized by altered basal metabolism.

The methods described herein can be used to determine the type and/or origin of cells found within the body by assessing Raman scattering characteristics of the cells and comparing them with the known Raman scattering characteristics of various cell types. In this way, the origin of a cancerous metastasis can be determined or migration of non-cancerous cells from a body location at which they normally occur to an abnormal body location can be detected.

The cells analyzed as described herein can be substantially any cells that can be obtained from, or accessed, in a mammal such as a human. Such cells can be cells obtained from a body fluid (e.g., urine, saliva, sputum, feces, blood, mucus, pus, semen, and fluid expressed from a wound or vaginal fluid), cells obtained by rinsing a body surface (e.g., a bronchial or peritoneal lavage); cells of a fresh tissue sample (e.g., scraped, biopsied, or surgically removed tissue), cells of paraffin-embedded or otherwise archived tissue samples, or cells that are examined in vivo in the mammal. The cells can be individual cells, clumps of cells, or cells that exist in a matrix of other cells and/or extracellular matrix.

Cells to be analyzed as described herein should be placed on and secured to a surface to prevent movement during analysis, unless the cells tend to adhere to the surface on their own. This is particularly important if Raman spectroscopy and light microscopy data are to be combined, because it is important to be able to correlate the microscopic characteristics of the cells, as directly or indirectly (e.g., using computer-processed or -stored image data) observed with the Raman scattering exhibited by the same cells. Cells can be secured or fixed on a surface using substantially any known technique, and any reagents known to exhibit strong Raman scattering at the RS values disclosed herein should be avoided or accounted for in scattering intensity determinations. Cells can be secured or fixed as individual cells on a substrate, as a substantially flat layer or slice of cells on a substrate, or as a three-dimensional mass of cells. When a secured or fixed cell preparation includes cells at different elevations above the surface of the substrate, spatial analysis of the preparation is possible using known adaptations to light microscopy and Raman scattering methods. By way of example, Raman scattering can be correlated with height above the substrate by assessing Raman scattering using different planes of focus. Information obtained at the various planes can be reconstructed (e.g., using a computer for storage and display of the information) to provide a two- or three-dimensional representation of the sample.

Raman scattering analysis can be assessed for cells in vivo, for example using an insertable and removable fiber-optic probe, such as the fiberscope described in U.S. Pat. No. 6,788,860. The probe can be fixed in place relative to the cells being assessed using known methods, and such fixation should employed if reproducible accessing of the cells is desired. For example, if cells are to be assessed in vivo to determine their disease status and cells determined to be diseased are thereafter to be ablated by delivery thereto of intense laser illumination, then it is important that the probe not be displaced relative to the cells during the interval between determination of disease status and ablation.

Combined Raman Spectroscopic Analysis and Visible Light Microscopy

Cellular imaging based on optical spectroscopy, in particular Raman spectroscopy, can provide a clinician with important information. Such techniques can be performed ex vivo (e.g., on raw, fixed, or mounted cells, tissues, or biopsies, or on a raw or purified body fluid) or in vivo (e.g., using endoscopic techniques). Molecular imaging simultaneously provides chemical morphological information (i.e., size, shape and distribution) for molecular species present in the sample. Using Raman spectroscopic imaging, a trained clinician can determine the disease state of a tissue or cellular sample based on recognizable changes in chemical morphology without the need for sample staining or modification.

By contrast, visible light microscopy offers the trained clinician only physical morphological and structural clues regarding the disease state of the cells or tissue being examined. Use of colored or fluorescent dyes can provide limited information regarding the cell surface or internal constituents of the cells, and can aid in determining the identity (i.e., cell or tissue type) or biochemical makeup of the cells. However, many staining reagents and methods can alter the morphology and/or structure of cells and tissue, thereby destroying useful information even as they reveal other information. Furthermore, many staining reagents and methods cannot practically be used for in vivo imaging or imaging of cells.

Combining Raman spectroscopy and visual light microscopy techniques enhances the usefulness of each by adding context to the information generated by the separate methods. Thus, physical, morphological, and structural information derivable from microscopic examination can be understood in the context of the biochemical makeup of the corresponding cellular materials and Raman scattering-based clues to the disease state and/or metabolic state of the cells being examined. If desired, staining or labeling reagents can be used in combination with Raman scattering and light microscopy in order to yield further information about the cells. By way of example, the presence of micrometastases in lymph nodes draining bladder tissue provide important information regarding the stage and metastatic potential of a bladder tumor, which information can be used to select an appropriately aggressive anti-cancer treatment. However, differentiating between bladder cells and other cells which may occur in a lymph node can be difficult, as can differentiating between cancerous and non-cancerous bladder cells. Using the methods described herein, bladder cells in a lymph node can be identified using microscopic techniques (e.g., using a bladder cell-specific staining reagent such as a labeled monoclonal antibody) and Raman scattering spectroscopy can be used to assess the cancerous state of any bladder cells identified in the lymph node. Alternatively, Raman scattering techniques can be used both to identify cell type (i.e., by assessing characteristic Raman spectral properties of cells) and to determine the disease status of the cells that are present.

Substantially any Raman spectrometer capable of defining, detecting, or capturing data from cell- and tissue-scale samples can be used to generate the Raman scattering data described herein. Likewise, substantially any light microscopy instrument can be used to generate visible light microscopy information. In circumstances in which positions of cells in the sample can be correlated (e.g., by analysis of cell position and/or morphology or by analysis of indicia on or shape of the substrate), it is not necessary that the Raman and microscope be integrated. In such circumstances, the data collected from each instrument can be aligned from separate observations. Preferably, however, a single instrument includes the Raman spectroscopy and light microscopy functionalities, is able to perform both analyses on a sample within a very short time period (e.g., less than one hour, preferably less than 10 minutes or 1 minute), and is able to correlate the spatial positions assessed using the two techniques. Information gathered using such an instrument can be stored in electronic memory circuits, processed by a computer, and/or displayed together to provide a depiction of the cell sample that is more informative than the separate depictions of the information obtained by the two techniques. A suitable example of equipment having these characteristics is the FALCON (RTM) RMI microscope available from ChemImage Corp. (Pittsburgh, Pa.). Suitable instruments are also described in U.S. Pat. No. 6,002,476 and in co-pending U.S. patent application Ser. No. 09/619,371.

A visible light microscope is not the only instrument which can be used in conjunction with a Raman spectrometer to analyze cells as described herein. Substantially any spectroscopic instrument can be used cooperatively with a Raman spectrometer, so long as at least some portion of the field of view of each instrument can be correlated with a portion of the field of view of the other. By way of example, a Raman spectrometer can be coupled with both a visible light microscope and a fluorescent spectrometer, using the same optics (e.g., as in the FALCON™ microscope system of ChemImage Corp.) or different optical paths. Data collected using the Raman and fluorescent spectrometers can be combined with visual data collected using the visible light microscope, for example by i) correlating the intensity of red shading of one or more portions of the visible microscopic field with the intensity of Raman scattered light at a selected RS value originating from the portion(s) and ii) correlating the intensity of green shading of one or more portions of the visible microscopic field with the intensity of fluorescent light at a particular wavelength emitted from the portion(s). By combining the information obtainable from multiple spectroscopic instruments, multiple optical properties of cells can be determined. A non-limiting list of such optical properties include absorbance, fluorescence, Raman scattering, and polarization characteristics. These devices and techniques can also be used to determine morphological and kinetic properties of cells, such as their shape and movement. Each of the properties thus determined can be used to assess the cancerous state of the cell, for example by comparison with properties of cells known to be cancerous or non-cancerous.

An example of a probe suitable for in vivo analysis of cells in a mammal is described in co-pending U.S. patent application Ser. No. 10/184,580 (publication no. U.S. 2003/0004419 A1, which is incorporated herein by reference). The tip of the probe can be inserted near or against a tissue of interest and Raman scattering and visible microscopic image data can be collected therefrom, optionally at various discrete depths using focusing techniques and/or at various RS values. Substantially any fiber optic or other optical probe that can deliver irradiation to a tissue in vivo and collect Raman light scattered therefrom can be adapted to an appropriate Raman spectrometer to perform the methods described herein. The probe preferably also includes an optical channel (e.g., a common optical fiber or a separate one) to facilitate microscopic imaging of the same tissue for which Raman spectroscopy is performed.

Information generated from Raman spectroscopy and/or light microscopy as described herein can be stored in electronic memory circuits, such as those of a computer, for storage and processing. A wide variety of data analysis software packages are commercially available. Suitable types of software include chemometric analysis tools such as correlation analysis, principle component analysis, factor rotation such as multivariate curve resolution, and image analysis software. Such software can be used to process the Raman scattering and/or visible image data to extract pertinent information that might otherwise be missed by univariate assessment methods.

Images of spectral information obtained from a single field of view of a sample can be combined in a straightforward manner if the images are obtained using the same optical path. For instance, a multimodal imaging instrument such as the FALCON™ Raman imaging microscope of ChemImage Corp. (Pittsburgh, Pa.) can be used to obtain Raman, fluorescent, and visible light reflectance image data from a sample using the same field of view and substantially the same optical path). Spatial alignment of spectral images can be as simple as overlying the spectral images from a single field of view, optionally with minor automated or manual alignment of image features or with software alignment using a program such as CHEMIMAGE XPERT (TM, ChemImage Corp., Pittsburgh, Pa.).

### EXAMPLE

The invention is now described with reference to the following Example. This Example is provided for the purpose of illustration only, and the invention is not limited to this Example, but rather encompasses all variations which are evident as a result of the teaching provided herein.

Raman molecular imaging (RMI) was used to distinguish cancerous and non-cancerous bladder cancer cells to demonstrate that RMI is useful for detection of bladder cancer.

RMI is an innovative technology that combines the molecular chemical analysis capacity of Raman spectroscopy with the power of high definition digital image microscopic visualization. This platform enables physicians and their assistants to identify both the physical architecture and molecular environment of cells in a urine sample and can complement, or even replace, current histopathological methods.

The data presented in this example demonstrate that the Raman scattering signal from bladder cancer tissue and cells voided in the urine can be identified and be distinguished from normal bladder tissue and cells. Detectable differences between high and low grade tumor cells were observed. These data establish that RMI signatures of bladder cancer cells are viable for discriminating high and low grades of bladder cancer, so that the disease can be detected in its earliest stages. These results demonstrate that RMI can be used as a non-invasive screening tool for detection of bladder cancer, for example in high risk populations (e.g., smokers over 40 years of age).

The experimental data presented below were derived from measurements made using a FALCON™ RMI microscope obtained from ChemImage Corp. (Pittsburgh, Pa.). The FALCON™ system uses 532 nanometer laser light to illuminate a sample over a wide field and collects Raman image data at multiple Raman shift (RS) values using a liquid crystal tunable filter (LCTF) unit equipped with a cooled charge-coupled device (CCD) array detector. This system is capable of collecting Raman spectra of the entire field of view and simultaneously acquiring Raman imaging spectral data and dispersive spectral data, as described in U.S. Pat. No. 6,717,768. Those features permit selection between full-field imaging and full-field collection of spectral data using a single set of optics. Data was processed using the CHEMIMAGE ANALYZE™ 6.0 spectral image processing software obtained from ChemImage Corp., applying standard techniques for signal processing and multivariate spectral data reduction techniques.

Samples were derived from anatomical pathology specimens retained in a cryogenic tissue bank. Sections of tissue samples embedded in TISSUE-TEK OCT (RTM) (10.24% w/w polyvinyl alcohol; 4.26% w/w polyethylene glycol; 85.50% w/w non-reactive ingredients; obtained from Saura Finetek U.S.A., Torrence, Calif.) were cut using a cryomicrotome at a thickness of 10 micrometers and placed on optical quality fused silica microscope slides. Excess OCT was removed with deionized water, and slides were air dried.

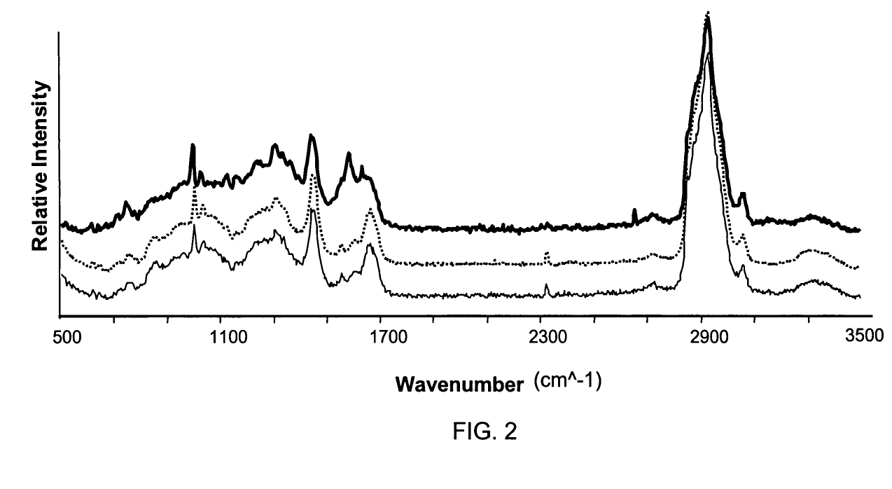

FIG. 2 shows the Raman spectra of bladder mucosal cells obtained from two patients not afflicted with bladder cancer (thin solid and dotted lines in FIG. 2) and from one patient afflicted with bladder carcinoma (thick solid line in FIG. 2).

The Raman spectra shown in FIG. 2 indicate the reproducibility of spectra for normal (non-cancerous) mucosa. Significant differences between the Raman spectra of the normal samples and the mucosal sample obtained from the patient afflicted with bladder carcinoma can be seen, for example at Raman shift values in the range from about 1000 to 1650 cm⁻¹, and more pronounced in the region from about 1525 to 1650 cm⁻¹. These data indicate that bladder carcinoma cells can be differentiated from normal bladder mucosal cells by RMI.

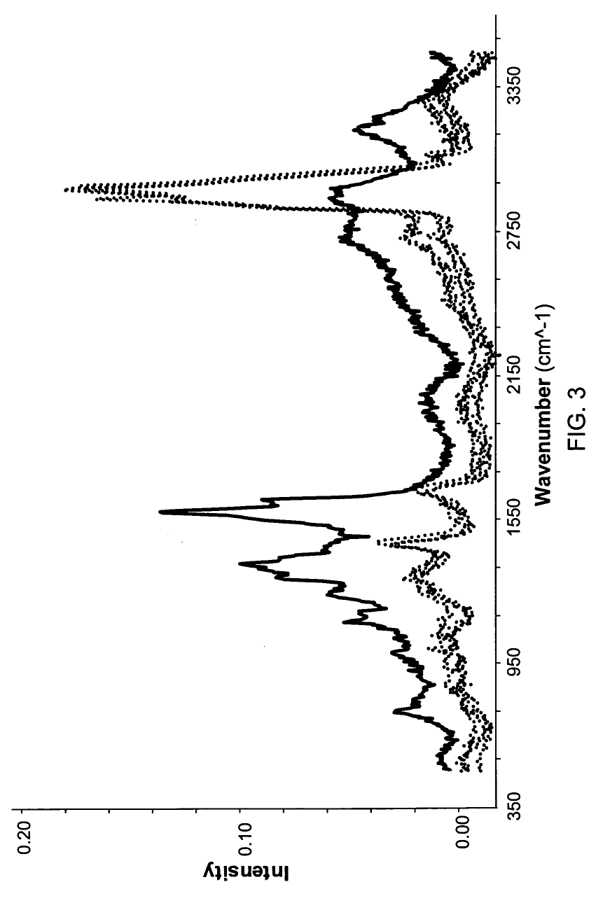

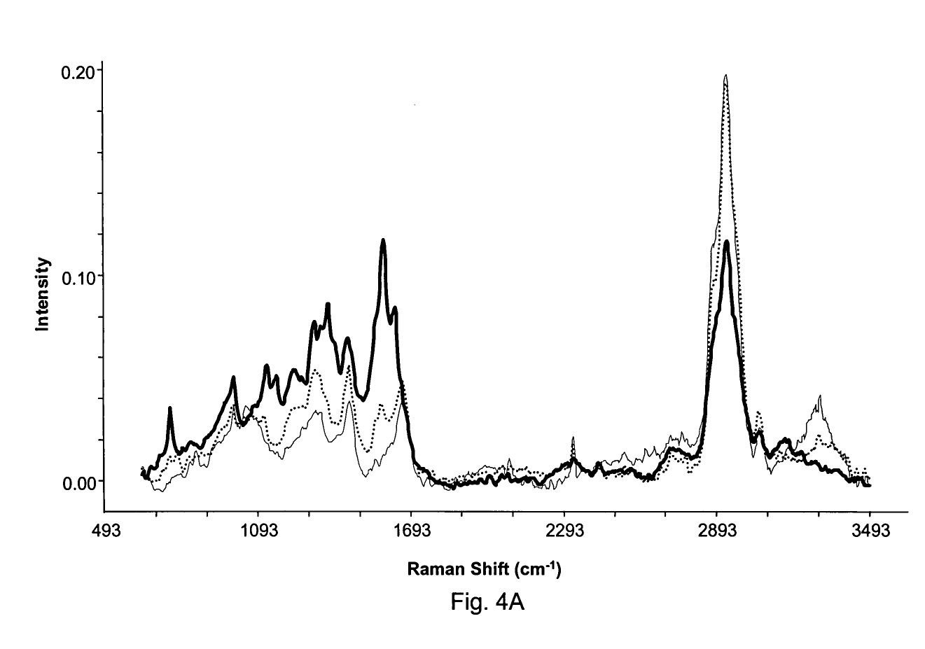

FIG. 3 shows the differences between the Raman spectra for three normal bladder mucosa tissue samples and a grade 3 transitional cell carcinoma (TCC) tissue. Significant Raman scattering intensity differences (between normal and TCC bladder mucosa tissues) were observed at Raman shifts of about 1000, 1250, 1370, and 1584 cm⁻¹.

Smears of cells from the grade 3 TCC bladder mucosa were prepared by manually pressing the tissue against the slide and dragging it across the aluminum surface. Raman spectra of the smears were obtained, and the spectra were found to be reproducible among the smears prepared. Furthermore, the Raman spectra obtained using smears were virtually identical to the Raman spectra obtained using intact tissue samples. These results indicate that the RMI method is not highly sensitive to the method used to prepare the cells for imaging, meaning that relatively simple cytological preparative methods can be employed.

Once the ability to recognize reproducible results from tissues had been established, single cell monitoring methods, investigating cells shed in urine, were developed. Red blood cells (RBCs) and other suspended or soluble substances present in normal urine can interfere with RMI. For example, RBCs exhibit Raman scattering peaks at Raman shifts (wavenumber values) of 1380 cm⁻¹ and 1590 cm⁻¹. It was found to be desirable to rinse cells (e.g., with distilled water) prior to RMI in order to avoid interference from RBCs, cell and tissue debris, and other potentially interfering substances in urine. This was performed by collecting cells from urine samples by centrifugation, rinsing the collected cells with distilled, deionized water, again centrifuging, and re-suspending the cells. A drop of the cell suspension was placed on an aluminum-coated microscope slide and smeared using another slide. In tissue sections, paraffin should also be removed as thoroughly as possible.

Microscopic inspection of cells obtained from urine samples indicated that there were white blood cells (WBCs) present. Raman spectra of WBCs and transitional epithelium are distinguishable from the Raman spectra of normal bladder mucosal cells. Nonetheless, it is preferable to remove WBCs from urine samples prior to assessing Raman scattering data from the remaining cells. Even if WBCs are not removed from the sample, their morphology and Raman scattering characteristics can be used to distinguish them from other cells in the sample.

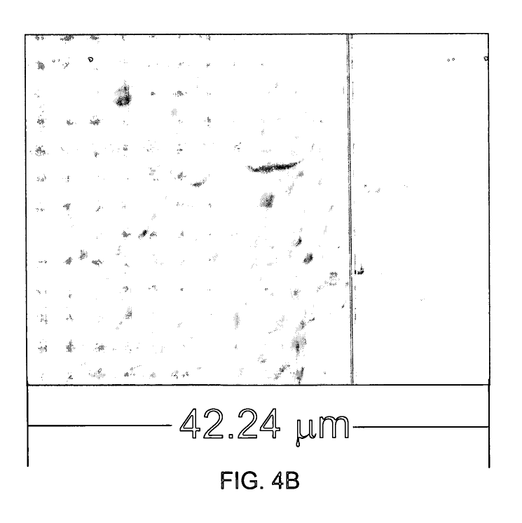

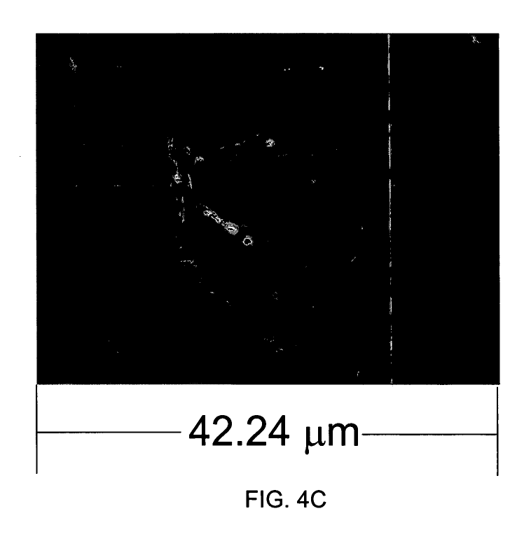

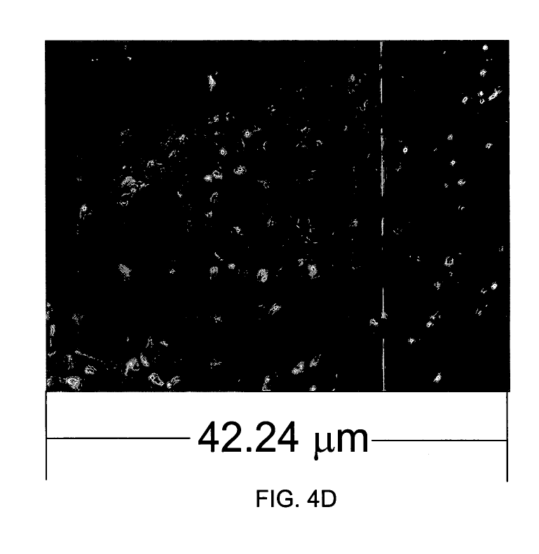

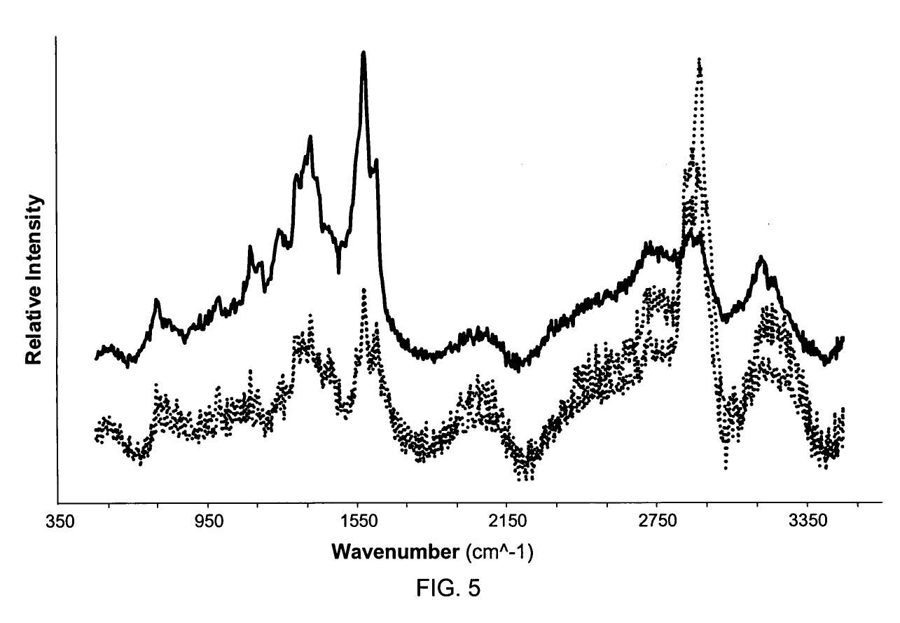

The Raman spectrum of normal bladder cells obtained from urine differed significantly from the Raman spectra of low and high grade malignant bladder cells obtained from urine. These results are shown in FIG. 4. FIGS. 4B, 4C, and 4D are brightfield micrographs of normal, low grade tumor, and high grade tumor bladder cells, respectively. FIG. 4A shows the Raman spectra of these cells. Significant Raman scattering intensity differences (between normal and tumor cells) are observed at approximate Raman shift values of 2900, 1584, 1370, and 1250 cm⁻¹. The high and low grade cells have similar spectra, but they exhibit small differences in some spectral regions. As shown in FIG. 5, these differences appear to be significant at Raman shift values of about 2900, 1584, 1370, 1250, and 1100 cm^{-1.}

The results described above include Raman spectra which extend over both the so called “fingerprint region” (roughly 280-1800 cm⁻¹) and the “CH” region (roughly between 2750 and 3200 cm⁻¹). The CH region is often neglected in Raman spectroscopy of biological samples because of the purported lack of specificity and biological relevance of Raman spectral information obtained for this region. The data presented in these figures demonstrate that the proportion of signal in the CH band relative to the fingerprint region varies between cancer and normal samples. Cancer samples tend to have proportionally more scatter in the fingerprint region. By normalizing the spectra such that the area under each curve is the same, this is evident by comparing the heights of the peaks in the fingerprint region to the peak in the CH region, as shown in FIGS. 2 and 3. The value of including the CH region in Raman analysis extends to the imaging paradigm where Raman images of a sample taken in the CH region can be used to ratiometrically standardize fingerprint region information to allow comparison of samples and distinction of signals which represent cancer.

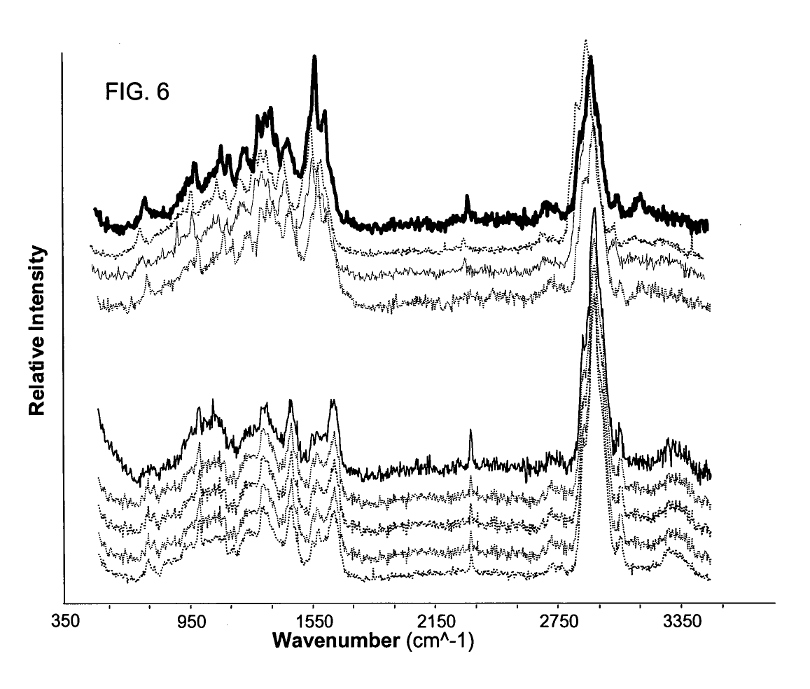

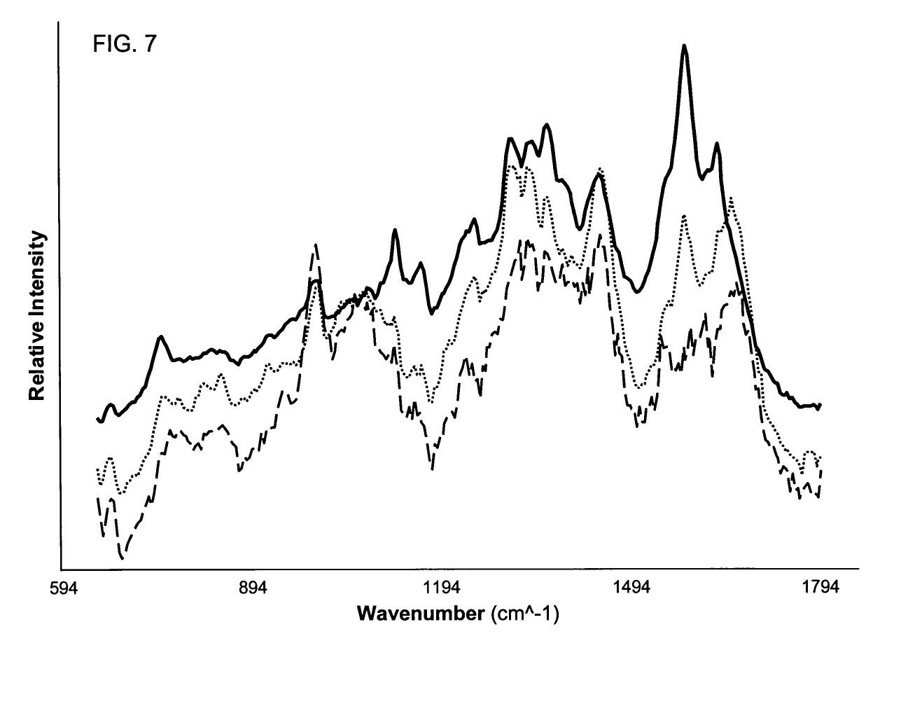

The results shown in FIGS. 4, 5, 6, and 7 demonstrate that Raman scattering data generated as described herein can be used to differentiate bladder cancer cells of different grades. The methods described herein can therefore be used to assess cancer grade in patients and to inform treatment decisions. Combined with superficial and/or microscopic visual analysis, the tumor can be more accurately and thoroughly characterized than was previously possible. The grade determination can also be made more quickly than was previously possible.

The disclosure of every patent, patent application, and publication cited herein is hereby incorporated herein by reference in its entirety.

While this invention has been disclosed with reference to specific embodiments, it is apparent that other embodiments and variations of this invention can be devised by others skilled in the art without departing from the true spirit and scope of the invention. The appended claims include all such embodiments and equivalent variations.

1. A method of assessing the cancerous state of a human bladder cell, the method comprising
irradiating the cell with substantially monochromatic light having a wavelength not greater than 695 nanometers,
assessing Raman scattered light emitted by the cell at a Raman shift (RS) value selected from the group consisting of RS values in the range from 280 to 1800 cm⁻¹ and RS values in the range from 2750 to 3200 cm⁻¹, and
comparing the intensity of the Raman scattered light emitted by the cell with a reference value corresponding to the intensity of Raman scattered light emitted by a reference bladder cell,
whereby a difference between the intensity of the Raman scattered light emitted by the cell and the reference value is indicative of the cancerous state of the cell.

2. The method of claim 1, wherein the cancerous state of the reference cell is known.

3. The method of claim 1, comprising assessing Raman scattered light emitted by the cell at a plurality of the RS values.

4. The method of claim 3, comprising comparing a spectrum of Raman scattered light emitted by the cell over a range of the RS values with a reference spectrum corresponding to Raman scattered light emitted by the reference cell over the same range.

5. The method of claim 4, wherein the range of RS values includes 1500 to 1650 cm⁻¹.

6. The method of claim 5, wherein the range of RS values includes 600 to 1800 cm⁻¹.

7. The method of claim 1, comprising assessing Raman scattered light emitted by the cell at an RS value in the range from 1500 to 1650 cm⁻¹.

8. The method of claim 1, comprising assessing Raman scattered light emitted by the cell at an RS value of about 1584 cm⁻¹.

9. The method of claim 1, wherein the wavelength of the substantially monochromatic light is about 532 nanometers.

10. The method of claim 1, wherein the bandwidth of the substantially monochromatic light is not greater than 0.25 nanometer.

11. The method of claim 10, wherein the wavelength of the substantially monochromatic light is about 532 nanometers.

12. The method of claim 1, wherein the substantially monochromatic light is generated by a laser.

13. The method of claim 1, wherein the bladder cell is substantially separated from red blood cells prior to assessing Raman scattered light emitted by the cell.

14. The method of claim 1, wherein the bladder cell is substantially separated from debris prior to assessing Raman scattered light emitted by the cell.

15. The method of claim 14, wherein the bladder cell is substantially separated from red blood cells prior to assessing Raman scattered light emitted by the cell.

16. The method of claim 1, wherein the bladder cell is obtained from a tissue biopsy.

17. The method of claim 1, wherein the bladder cell is obtained from a lymph node.

18. The method of claim 1, wherein the bladder cell is obtained from a body fluid.

19. The method of claim 18, wherein the body fluid is urine.

20. The method of claim 11, wherein the bladder cell is obtained from a fluid that has been contacted with the bladder.

21. The method of claim 20, wherein the fluid is selected from the group consisting of a bladder imaging contrast fluid and a bladder lavage fluid.

22. The method of claim 1, wherein the bladder cell is irradiated in vivo.

23. The method of claim 1, wherein the bladder cell is irradiated using a laser illumination source for illuminating an area of a sample that includes the cell.

24. The method of claim 23, wherein Raman scattered light emitted by the bladder cell is assessed by
collecting a Raman spectrum of scattered light from the illuminated area of the sample using an objective and producing a collimated beam therefrom;
filtering the collimated beam using an Evans split-element liquid crystal tunable filter; and
collecting the filtered, collimated beam using a detector.

25. The method of claim 24, further comprising storing the output from the detector in a computer memory.

26. The method of claim 1, wherein Raman scattered light emitted by the bladder cell is assessed at two sampled RS values, and the ratio of intensities of the Raman scattered light emitted by the cell at the sampled RS values is compared with a reference ratio value corresponding to the ratio of intensities of Raman scattered light emitted by the reference bladder cell at the sampled RS values.

27. The method of claim 1, wherein Raman scattered light emitted by the bladder cell is separately assessed at a plurality of locations on the cell.

28. The method of claim 27, wherein the locations are substantially non-overlapping.

29. The method of claim 27, wherein Raman scattered light emitted by the cell is substantially simultaneously assessed at each of the plurality of locations.

30. The method of claim 27, wherein the plurality of Raman light scattering assessments is made in parallel using an array of detectors.

31. The method of claim 1, wherein Raman scattered light emitted by each of a plurality of bladder cells in a sample is separately assessed.

32. A method of generating an image informative of the cancerous state of a human bladder cell, the method comprising
irradiating a sample that includes the cell with substantially monochromatic light having a wavelength not greater than 695 nanometers,
assessing Raman scattered light emitted by the cell at an RS value selected from the group consisting of RS values in the range from 280 to 1800 cm⁻¹ and RS values in the range from 2750 to 3200 cm⁻¹,
generating an image of the Raman scattered light emitted by the cell, and
combining a visual image of the cell and the image of Raman scattered light emitted by the cell,
wherein the combined image is informative of the cancerous state of the cell.

33-34. (canceled)

35. A method of generating an image informative of the cancerous state of a human bladder cell, the method comprising
irradiating a sample that includes the cell with substantially monochromatic light having a wavelength not greater than 695 nanometers,
separately assessing Raman scattered light emitted at a plurality of locations in the sample at an RS value selected from the group consisting of RS values in the range from 280 to 1800 cm⁻¹ and RS values in the range from 2750 to 3200 cm⁻¹,
generating an image of the Raman scattered light emitted at the locations in the sample, and
combining a visual image of the sample and the image of the Raman scattered light emitted at the locations in the sample,
whereby the resulting image is informative of the cancerous state of the individual cells.

36-43. (canceled)

44. A method of ablating a cancerous bladder cell in a human, the method comprising
irradiating bladder cells in the human with substantially monochromatic light having a wavelength not greater than 695 nanometers,
assessing Raman scattered light emitted by an individual bladder cell at a Raman shift (RS) value selected from the group consisting of RS values in the range from 280 to 1800 cm⁻¹ and RS values in the range from 2750 to 3200 cm⁻¹,
comparing the intensity of the Raman scattered light emitted by the individual cell with a reference value corresponding to the intensity of Raman scattered light emitted by a reference bladder cell in order to indicate whether the bladder cell is cancerous, and
ablating the individual cell if it is cancerous.
